(19) United States
(12) Patent Application Publication
(10) Pub. No.: US 2025/0249131 A1
(43) Pub. Date: Aug. 7, 2025

(54) SACCHARIDE-BASED MULTI-ARM POLYMERS FOR MEDICAL APPLICATIONS AND HYDROGELS FORMED FROM SAME

(71) Applicant: Boston Scientific Scimed, Inc., Maple Grove, MN (US)

(72) Inventors: Joseph Thomas Delaney, JR., Minneapolis, MN (US); Liming Crandall, Lino Lakes, MN (US); Yen-Hao Hsu, Shrewsbury, MA (US); Cristian Parisi, Amherst, MA (US); Heather Cirka, Hudson, MA (US); Nicolas Ball-Jones, Minneapolis, MN (US); Rick Dorn, Plymouth, MN (US)

(73) Assignee: Boston Scientific Scimed, Inc., Maple Grove, MN (US)

(21) Appl. No.: 19/039,070

(22) Filed: Jan. 28, 2025

Related U.S. Application Data
(60) Provisional application No. 63/548,693, filed on Feb. 1, 2024.

(51) Int. Cl. A61K 49/04; A61M 3/00

(52) U.S. Cl. CPC A61K 49/0442; A61K 49/0457; A61M 3/005

(57) ABSTRACT

In some aspects, the present disclosure relates to reactive multi-arm polymers that comprise (a) a core region, (b) multiple polymer arms, each polymer arm comprising at least one polymer segment and having a first end and a second end, the first end linked to the core region and (c) at least one first reactive moiety linked to the second end of each of the polymer arms, wherein the core region comprises a residue of a saccharide-based compound in which hydroxyl groups of a monosaccharide, disaccharide or oligosaccharide are replaced by C₃-C₁₀-hydroxyalkyloxy groups. In other aspects the present disclosure provides crosslinked hydrogels formed by covalently crosslinking such reactive multi-arm polymers with multifunctional crosslinking compounds that comprise a plurality of second reactive moieties that are reactive with the first reactive moieties of the reactive multi-arm polymer, as well as systems for forming such crosslinked hydrogels.

### CROSS-REFERENCE TO RELATED APPLICATIONS

The application claims the benefit of U.S. Provisional Patent Application Ser. No. 63/548,693 filed on Feb. 1, 2024, the disclosure of which is incorporated herein by reference.

### FIELD

The present disclosure relates to saccharide-based multi-arm polymers and to crosslinkable systems for forming hydrogels, among other aspects. The hydrogels and crosslinkable systems for forming the same are useful, for example, in various medical applications.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups will rapidly react with the trilysine crosslinker in vivo to form a hydrogel. The hydrogels breakdown in-vivo over the course of ca. 6-9 months. The breakdown occurs primarily through the hydrolysis of the ester linkages on the glutarate groups.

### SUMMARY

The present disclosure provides alternative multi-arm polymers for use in hydrogel forming products as well as other medical applications. As used herein, a “hydrogel” is a crosslinked polymer that contains water or can absorb water but does not dissolve when placed in water.

In some aspects, the present disclosure relates to reactive multi-arm polymers that comprise (a) a core region, (b) multiple polymer arms, each polymer arm comprising at least one polymer segment and having a first end and a second end, the first end linked to the core region and (c) at least one first reactive moiety linked to the second end of each of the polymer arms, wherein the core region comprises a residue of a saccharide-based compound in which hydroxyl groups of a monosaccharide, disaccharide or oligosaccharide are replaced by C₃-C₁₀-hydroxyalkyloxy groups.

In some embodiments, the C₃-C₁₀-hydroxyalkyloxy groups are C₃-C₁₀-monohydroxyalkyloxy groups. In some of these embodiments, the C₃-C₁₀-monohydroxyalkyloxy groups are linear C₃-C₁₀-monohydroxyalkyloxy groups and wherein a hydroxyl group is positioned at a terminal carbon atom of each of the linear C₃-C₁₀-monohydroxyalkyloxy groups.

In some embodiments, which can be used in conjunction with the above aspects and embodiments, the C₃-C₁₀-hydroxyalkyloxy groups are C₃-C₁₀-dihydroxyalkyloxy groups. In some of these embodiments, the C₃-C₁₀-dihydroxyalkyloxy groups are linear C₃-C₁₀-dihydroxyalkyloxy groups having two hydroxyl groups, and the two hydroxyl groups are vicinal diols with a first hydroxyl group positioned at a terminal carbon of each of the linear C₃-C₁₀-dihydroxyalkyloxy groups and a second hydroxyl group positioned at a carbon atom adjacent to the terminal carbon atom.

In some embodiments, which can be used in conjunction with the above aspects and embodiments, the first reactive moieties are linked to the polymer segments through a hydrolysable linkage.

In some embodiments, which can be used in conjunction with the above aspects and embodiments, the first reactive moiety is selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, reactive moieties that comprise oxyamine groups, and reactive moieties that comprise acrylate groups.

In some embodiments, which can be used in conjunction with the above aspects and embodiments, the polymer segment is selected from polyalkylene oxide segments, polyester segments, polyoxazoline segments, polydioxanone segments, and polypeptide segments.

In some embodiments, which can be used in conjunction with the above aspects and embodiments, the polymer segment contains between 10 and 1000 monomer residues.

In other aspects, the present disclosure pertains to systems for forming hydrogel compositions, which systems comprise (a) the reactive multi-arm polymer in accordance with the above aspects and embodiments, and (b) a multifunctional crosslinking compound that comprises a plurality of second reactive moieties that are reactive with the first reactive moieties of the reactive multi-arm polymer.

In some embodiments, the plurality of second reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, reactive moieties that comprise oxyamine groups, and reactive moieties that comprise acrylate groups.

In some embodiments, which can be used in conjunction with the above aspects and embodiments, the multifunctional crosslinking compound is an iodinated multifunctional crosslinking compound. In some of these embodiments, the iodinated multifunctional crosslinking compound comprises an iodinated aromatic group.

In some embodiments, which can be used in conjunction with the above aspects and embodiments, the system further comprises a delivery device that is configured to simultaneously deliver the reactive multi-arm polymer and the multifunctional crosslinking compound to a patient under conditions where the reactive multi-arm polymer covalently crosslinks with the multifunctional crosslinking compound.

In other aspects, the present disclosure pertains to crosslinked hydrogels formed by covalently crosslinking (a) a reactive multi-arm polymer in accordance with the above aspects and embodiments with (b) a multifunctional crosslinking compound that comprises a plurality of second reactive moieties that are reactive with the first reactive moieties of the reactive multi-arm polymer.

In some embodiments, the plurality of second reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, reactive moieties that comprise oxyamine groups, and reactive moieties that comprise acrylate groups.

In some embodiments, which can be used in conjunction with the above aspects and embodiments, the multifunctional crosslinking compound is an iodinated multifunctional crosslinking compound. In some of these embodiments, the iodinated multifunctional crosslinking compound comprises an iodinated aromatic group.

In some embodiments, which can be used in conjunction with the above aspects and embodiments, the crosslinked hydrogel is in the form of crosslinked hydrogel particles. In some of these embodiments, the crosslinked hydrogel particles are contained in a syringe barrel.

The above and other aspects, embodiments, features and benefits of the present disclosure will be readily apparent from the following detailed description.

### BRIEF DESCRIPTION OF THE DRAWINGS

FIG. 1 schematically illustrates a method of forming a polyhydroxylated saccharide-based compound having hydroxyl-terminated pendant groups, in accordance with an embodiment of the present disclosure.

FIG. 2 schematically illustrates a method of forming a polyhydroxylated saccharide-based compound having hydroxyl-terminated pendant groups, in accordance with another embodiment of the present disclosure.

FIG. 3 schematically illustrates a method of forming a multi-arm polymer, in accordance with an embodiment of the present disclosure.

FIG. 4 schematically illustrates a method of forming a carboxyl-terminated multi-arm polymer, in accordance with an embodiment of the present disclosure.

FIG. 5 schematically illustrates a method of forming a succinimidyl-glutarate-terminated multi-arm polymer, in accordance with an embodiment of the present disclosure.

FIG. 6 schematically illustrates a method of forming a cyclooctyne-glutarate-terminated multi-arm polymer, in accordance with an embodiment of the present disclosure.

FIG. 7 schematically illustrates a method of forming a cyclooct-4-en-1-yl-glutarate-terminated multi-arm polymer, in accordance with an embodiment of the present disclosure.

FIG. 8 schematically illustrates a method of forming a tetrazinyl-terminated multi-arm polymer, in accordance with an embodiment of the present disclosure.

FIGS. 9A-9C schematically illustrate three crosslinking systems in accordance with embodiments of the present disclosure.

FIG. 10 schematically illustrates a delivery device, in accordance with an embodiment of the present disclosure.

FIG. 11 schematically illustrates a delivery device, in accordance with another embodiment of the present disclosure.

### DETAILED DESCRIPTION

Reactive multi-arm polymers for use in conjunction with the present disclosure include multi-arm polymers that comprise (a) a core region comprising a saccharide residue, (b) multiple polymer arms, each polymer arm comprising at least one polymer segment and having a first end and a second end, the first end linked to the core region and (c) at least one reactive moiety linked to the second end of each of the polymer arms.

Reactive moieties may be selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise oxyamine groups, reactive moieties that comprise ketone groups, reactive moieties that comprise aldehyde groups, and reactive moieties that comprise acrylate groups, among others.

Electrophilic groups include cyclic imide ester groups, including succinimide ester groups,

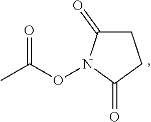

maleimide ester groups, glutarimide ester groups, diglycolimide ester groups, phthalimide ester groups, and bicyclo[2.2.1]hept-5-ene-2,3-dicarboxylic acid imide ester groups,

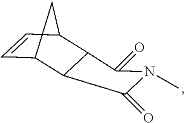

imidazole ester groups, imidazole carboxylate groups and benzotriazole ester groups, among other possibilities. Nucleophilic groups include primary amine groups, thiol groups and hydroxyl groups, among other possibilities. Diene containing groups include furan groups and tetrazine groups. Dienophile groups include norbornene groups and maleimide groups. Alkenyl-containing groups include vinyl groups, acryloyl groups, methacryloyl groups and strained alkene groups, for example, from cyclooct-4-en-1-yl groups,

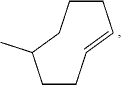

among other possibilities. Strained alkyne groups include cyclooctyne-containing groups such as (1R,8S,9s)-bicyclo[6.1.0]non-4-yn-9-yl groups,

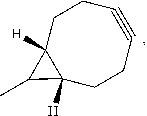

among other possibilities.

Reactive multi-arm polymers in accordance with the present disclosure may have three, four, five, six, seven, eight, nine, ten, eleven, twelve, thirteen, fourteen, fifteen, sixteen, seventeen, eighteen, nineteen, twenty, twenty-five, thirty or more polymer arms.

Polymer segments for the polymer arms may be selected from any of a variety of synthetic, natural, or hybrid synthetic-natural polymer segments. Examples of polymer segments include those that are formed from one or more monomers selected from the following: C₁-C₆-alkylene oxide monomers (e.g., ethylene oxide, propylene oxide, tetramethylene oxide, etc.), cyclic ester monomers (e.g. glycolide, lactide, β-propiolactone, β-butyrolactone, 7-butyrolactone, 7-valerolactone, 6-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), and 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Polymer segments for the polymer arms may be selected, for example, from the following polymer segments: polyether segments including poly(C₁-C₆-alkylene oxide) segments such as poly(ethylene oxide) (PEO) segments (also referred to as polyethylene glycol segments or PEG segments), poly(propylene oxide) (PPO) segments, poly(ethylene oxide-co-propylene oxide) segments, polyester segments including polyglycolide segments, polylactide segments, poly(lactide-co-glycolide) segments, poly(O-propiolactone) segments, poly(O-butyrolactone) segments, poly(7-butyrolactone) segments, poly(7-valerolactone) segments, poly(8-valerolactone) segments, and poly(F-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Each polymer segment for use in the multi-arm polymers of the present disclosure may contain between 2 and 1000 monomer units or more, for example, ranging anywhere from 2 to 5 to 10 to 30 to 70 to 100 to 300 to 700 to 1000 units (in other words, ranging between any two of the preceding values).

The core regions for the multi-arm polymers of the present disclosure comprise a residue of a saccharide, which has three or more hydroxyl groups. As explained below, each hydroxyl group can be used as a site for initiation or attachment of a polymer arm.

Saccharides for use herein include monosaccharides, disaccharides, oligosaccharides, and polysaccharides. Monosaccharides include trioses (having 3 carbon atoms), tetroses (having 4 carbon atoms), pentoses (having 5 carbon atoms), hexoses (having 6 carbon atoms), heptoses (having 7 carbon atoms), octoses (having 8 carbon atoms), nonoses (having 9 carbon atoms), and so forth. Disaccharides contain a combination of any two monosaccharide units, which may be the same or different, linked together by a glycosidic bond. Oligosaccharides contain a combination of between three and nine monosaccharide units, which may be the same or different, linked together by glycosidic bonds. Oligosaccharides thus include trisaccharides, tetrasaccharides, pentasaccharides, hexasaccharides, heptasaccharides, octasaccharides, and nonasaccharides. Polysaccharides contain a combination of ten or more monosaccharide units, which may be the same or different, linked together by glycosidic bonds.

Monosaccharides in open-chain form, particularly, trioses and tetroses, contain aldehyde and ketone groups that can be reduced to an alcohol using a suitable reducing agent, such as NaBH₄ or LiAlH₄. In these cases, for example, trioses can be subjected to reduction to afford a modified saccharide having three hydroxyl groups, and tetraoses can be subjected to reduction to afford a modified saccharide having four hydroxyl groups.

A particular example of a saccharide having three hydroxyl groups is a triose such as glyceraldehyde, which has been modified by a suitable reducing agent.

Particular examples of saccharides having four hydroxyl groups include tetroses, such as erythose or threose, which have been modified by a suitable reducing agent, and various unmodified pentoses, such as ribose, arabinose, xylose and lyxose.

Particular examples of saccharides having five hydroxyl groups include various unmodified hexoses such as glucose, allose, altrose, mannose, gulose, idose, galactose and talose.

Particular examples of saccharides having six hydroxyl groups include xylobiose (CAS #6860-47-5), among others.

Saccharides having seven hydroxyl groups include a number of disaccharides. Particular examples include rutinose (CAS #90-74-4), turanose (CAS #547-25-1), Blood group H disaccharide (CAS #16741-18-7), β-rutinose (CAS #26184-96-3), xylosucrose (CAS #512-66-3), sambubiose (CAS #26388-68-1), 2-deoxy-4-O-α-D-glucopyranosyl-D-arabino-hexose (55601-96-2), 6-O-β-D-xylopyranosyl-β-D-glucopyranose (133962-98-8), 6-O-(6-deoxy-α-L-mannopyranosyl)-D-galactopyranose (552-74-9), and methyl 6-O-β-D-glucopyranosyl-β-D-galactopyranoside (101144-26-7), among others.

Saccharides having eight hydroxyl groups include a number of disaccharides. Particular examples include allolactose (CAS #28447-39-4), cellobiose (CAS #528-50-7), isomaltose (CAS #499-40-1), lactose (CAS #63-42-3), lactulose (CAS #4618-18-2), maltose (CAS #69-79-4), melibiulose (CAS #111188-56-8), sophorose (CAS #534-46-3), sucrose (CAS #57-50-1), and trehalose (CAS #99-20-7), among others.

Particular examples of saccharides having nine hydroxyl groups include maltitol (CAS #585-88-6) and lactitol (CAS #585-86-4), among others.

Particular examples of saccharides having ten hydroxyl groups include mellitoside (19467-03-9), violanthin (40581-17-7), secoisolariciresinol diglucoside (158932-33-3), vicenin 2 (23666-13-9), isovitexin 7-glucoside (20310-89-8), and 4-methoxyphenyl O-α-D-galactopyranosyl-(1->4)O-β-D-galactopyranosyl-(1→4)-β-D-glucopyranoside (ACI) (898826-64-7), among others.

Saccharides having eleven hydroxyl groups include a number of trisaccharides. Particular examples include nigerotriose (CAS #23393-12-6), maltotriose (CAS #1109-28-0), melezitose (CAS #597-12-6), maltotriulose (CAS #1109-28-0), raffinose (CAS #512-69-6), and kestose (CAS #470-69-9), among others.

Saccharides having twelve hydroxyl groups include a number of trisaccharides. Particular examples include maltotriose, cellotriose, manninotriose, panose, and maltotriitol (CAS #32860-62-1).

Saccharides including many of those described above are widely available, have relatively low cost, and can be readily purified, for example, by crystallization.

In various embodiments, saccharides including those described above can be used to make multi-arm polymers with good architectural control.

For example, the hydroxyl groups of saccharides, including those described above, can be used as sites for initiation of polymer arms or attachment of polymer arms.

In some embodiments, the hydroxyl groups of saccharides, including those described above, can be used as sites for the formation of hydroxyl-containing pendant groups, which can subsequently be used as sites for initiation or attachment of polymer arms.

Such hydroxyl-containing pendant groups may act as hydroxyl-group spacers to ensure more equal reactivity of the hydroxyl groups, such hydroxyl-containing pendant groups may be provided to multiply the number of available hydroxyl groups, or both.

For example, in embodiments where a spacer is desired, hydroxyl-terminated pendant groups may be formed, each of which comprises a hydroxyl end group that is linked to a residue of the saccharide through an alkyl group, such as a C₃-C₁₀ alkyl group. For example, an w-halo-1-C₃-C₁₀-alkene, such as 3-halo-1-propene, 4-halo-1-butene, 5-halo-1-pentene, 6-halo-1-hexene, 7-halo-1-heptene, 8-halo-1-octene, 9-halo-1-nonene or 10-halo-1-decene, where “halo” represents a halogen group such as a bromo group, chloro group or iodo group (e.g., 3-bromo-1-propene, 4-bromo-1-butene, 5-bromo-1-pentene, 6-bromo-1-hexene, etc.) can be reacted with hydroxyl groups of a saccharide such as one of those described above, among others, resulting in a 2-propenyloxy group, a 3-butenyloxy group, a 4-pentenyloxy group, a 5-hexenyloxy group, a 6-heptenyloxy group, a 7-octenyloxy group, a 8-nonenyloxy group, or a 9-decenyloxy group at the site of each of the hydroxyl groups of the saccharide. In a subsequent step, the alkene group may be hydroxylated using hydroboration-oxidation to achieve anti-Markovnikov addition to the olefinic bonds, with the result being a C₃-C₁₀-hydroxyalkyloxy group, specifically, a 3-hydroxypropoxy group, a 4-hydroxybutoxy group, a 5-hydroxypentoxy group, a 6-hydroxyhexoxy group, a 7-hydroxyheptoxy group, a 8-hydroxyoctoxy group, a 9-hydroxynonoxy group, or a 10-hydroxydecoxy group, at the site of each of the hydroxyl groups of the saccharide. Hydroboration-oxidation may be achieved, for example, by reaction with a sterically demanding borane such as 9-Borabicyclo[3.3.1]nonane (9-BBN), followed oxidation with hydrogen peroxide (H₂O₂).

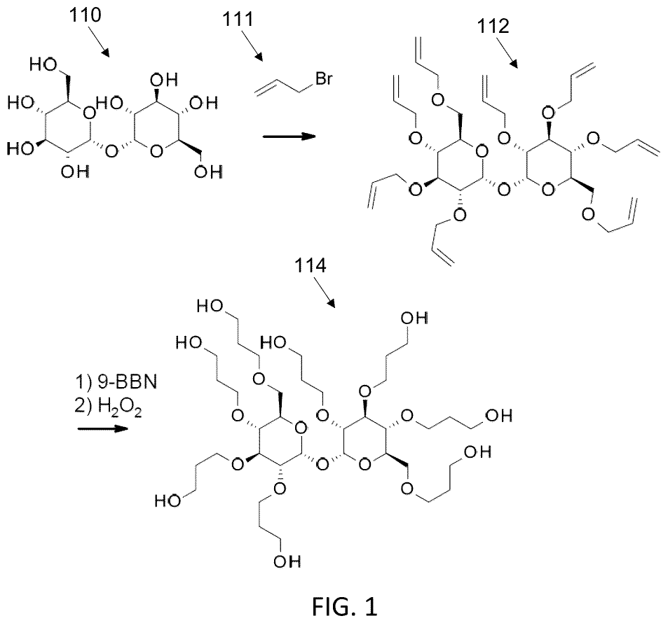

In a particular embodiment shown in FIG. 1, trehalose (110) is reacted with 3-bromo-1-propene (allyl bromide) (111) to form octakis(1-propenyl) trehalose (112), which is then converted to octakis(3-hydroxypropyl) trehalose (114) by hydroboration with 9-BBN, followed oxidation with H₂O₂.

In other embodiments, rather than hydroxylating the alkene groups by hydroboration-oxidation to achieve anti-Markovnikov addition to the olefinic bonds, the alkene groups can instead be dihydroxyled, for example, by Sharpless asymmetric dihydroxylation (also called the Sharpless bishydroxylation) in which the alkene is reacted with osmium tetroxide in the presence of a chiral quinine ligand to form a vicinal diol, thereby effectively doubling the number of hydroxyl groups associated with the saccharide. For example, after forming a 2-propenyloxy, 3-butenyloxy, 4-pentenyloxy, 5-hexenyloxy, 6-heptenyloxy, 7-octenyloxy, 8-nonenyloxy, or 9-decenyloxy group at the site of each of the hydroxyl groups of the saccharide as described above, the alkene groups can be dihydoxylated to form C₃-C₁₀-dihydroxyalkyloxy group, specifically, a 2,3-dihydroxypropoxy group, a 3,4-dihydroxybutoxy group, a 4,5-dihydroxypentoxy group, a 5,6-dihydroxyhexoxy group, a 6,7-dihydroxyheptoxy group, a 7,8-dihydroxyoctoxy group, a 8,9-dihydroxynonoxy group, or a 9,10-dihydroxydecoxy group at the site of each of the hydroxyl groups of the saccharide.

A similar result can be achieved by first reacting hydroxyl groups of a saccharide such as one of those described above, among others, with an acetal-protected vicinal diol halide, such as an acetal-protected 3-halo-1,2-propanediol group, an acetal-protected 4-halo-1,2-butanediol group, an acetal-protected 5-halo-1,2-pentanediol group, an acetal-protected 6-halo-1,2-hexanediol group, an acetal-protected 7-halo-1,2-heptanediol group, an acetal-protected 8-halo-1,2-octanediol group, an acetal-protected 9-halo-1,2-nonanediol group, or an acetal-protected 10-halo-1,2-decanediol group, wherein “halo” represents a halogen group such as a bromo, chloro or iodo group, for example, such as 4-(bromomethyl)-2,2-dimethyl-1,3-dioxolane (CAS #36236-76-7),

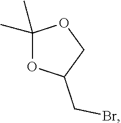

4-(2-bromoethyl)-2,2-dimethyl-1,3-dioxolane (CAS #89942-18-7),

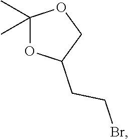

4-(3 bromopropyl)-2,2-dimethyl-1,3-dioxolane (CAS #251998-53-5), 4-(4-bromobutyl)-2,2-dimethyl-1,3-dioxolane, 4-(5-bromopentyl)-2,2-dimethyl-1,3-dioxolane, and so forth. Subsequently, the acetal protection is removed via acid catalyzed hydrolysis to yield a 2,3-dihydroxypropoxy group, a 3,4-dihydroxybutoxy group, a 4,5-dihydroxypentoxy group, a 5,6-dihydroxyhexoxy group, a 6,7-dihydroxyheptoxy group, a 7,8-dihydroxyoctoxy group, an 8,9-dihydroxynonoxy group, or a 9,10-dihydroxydecoxy group at the site of each of the hydroxyl groups of the saccharide.

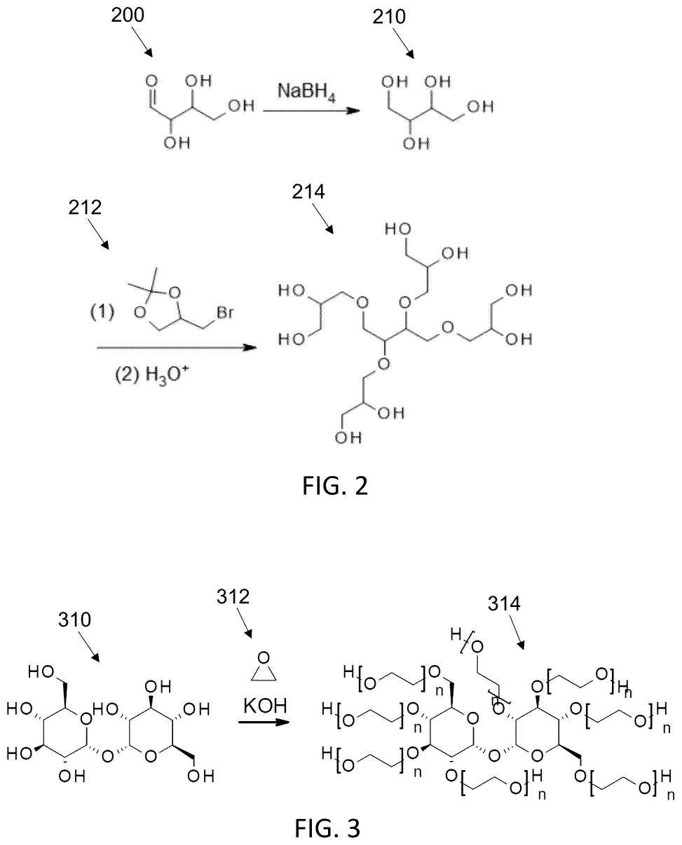

Referring now to FIG. 2, in a first step, a saccharide having three hydroxyl groups, specifically, erythose (200) is modified using a suitable reducing agent, such as NaBH₄, to convert the aldehyde group to a hydroxyl group as previously described. The resulting saccharide (210), which has four hydroxyl groups, is then reacted with an acetal-protected vicinal diol halide, specifically, 4-(bromomethyl)-2,2-dimethyl-1,3-dioxolane) (212), followed by removal of the acetal groups via acid catalyzed hydrolysis to yield a saccharide having eight hydroxyl groups, specifically, 1,2,3,4-tetra(2,3-dihydroxypropoxy)butane (214).

In further embodiments where a spacer as well as a hydrolysable ester linkage is desired, hydroxyl groups of saccharides, including those described above, among many others, may be reacted with a lactone (e.g., α-acetolactone, β-propiolactone, γ-butyrolactone, 6-valerolactone, F-caprolactone, etc.) to form hydroxyl-terminated pendant groups comprising a hydroxyl end group that is linked to a residue of the polysaccharide through an alkyl group (e.g., C₁-C₁₀ alkyl group) and a hydrolysable ester group at the site of each of the hydroxyl groups of the saccharide.

In each of the above embodiments, a polyhydroxylated saccharide-based compound is produced that comprises a plurality hydroxyl-containing pendant groups linked to a saccharide residue core.

In various embodiments, saccharides including those described above are used as multi-functional initiators for chain growth of polymer arms of a star polymer. For example, such saccharides can be used as initiators for ring-opening polymerization of ethylene oxide to form poly(ethylene oxide) (PEG) segments at the site of each of the hydroxyl groups of the saccharide.

In a particular embodiment shown in FIG. 3, commercially available trehalose (310) can be used as an octa-functional initiator by treating it with a base such as potassium hydroxide, followed by addition of ethylene oxide (312), which undergoes ring-opening polymerization. The polymerization process leads to poly(ethylene oxide) (PEG) chain growth at each of the eight hydroxyl groups of the trehalose, forming a hydroxy-terminated 8-arm-PEG (314) having a trehalose residue core. In FIG. 3, n is an integer representing the number of monomer units in each polymer segment shown. In other cases, only a single monomer unit may be present and n=1. By adjusting the stoichiometry of the initiator relative to the ethylene oxide, the final degree of polymerization is controlled.

The strategy shown in FIG. 3 is broadly applicable to other hydroxyl-containing compounds and can be used in conjunction with a wide range of saccharides and polyhydroxylated saccharide-based compounds, including those described above.

Moreover, the hydroxyl groups of saccharides and polyhydroxylated saccharide-based compounds, including those described above, can be used as points of attachment for preformed polymer segments. For example, carboxylic acid terminated polymer segments may be reacted with the hydroxyl groups to form ester linkages at the hydroxyl group sites.

In some embodiments, it may be desirable to react the terminal hydroxyl groups of polymer arms of multi-arm polymers, for example, the terminal hydroxyl groups of the polymer arms of the multi-arm polymer of FIG. 3, to form carboxyl groups, for example, in conjunction with providing the polymers with reactive moieties.

In particular embodiments, terminal hydroxyl groups of the polymer arms maybe reacted with a cyclic anhydride compound (e.g., glutaric anhydride, succinic anhydride, malonic anhydride, adipic anhydride, diglycolic anhydride, 1,3-acetonedicarboxylic acid anhydride, etc.) to form an acid-end-capped polymer segment such as a glutaric-acid-end-capped segment, a succinic-acid-end-capped segment, a malonic-acid-end-capped segment, an adipic-acid-end-capped segment, a diglycolic-acid-end-capped segment, a 1,3-acetonedicarboxylic-acid-end-capped segment, and so forth. The preceding cyclic anhydrides, among others, may be reacted with a hydroxy-terminated multi-arm polymer under basic conditions to form a carboxylic-acid-terminated multi-arm polymer in which the polymer arms comprise a carboxylic acid end group that is linked to the polymer arm through a hydrolysable ester group.

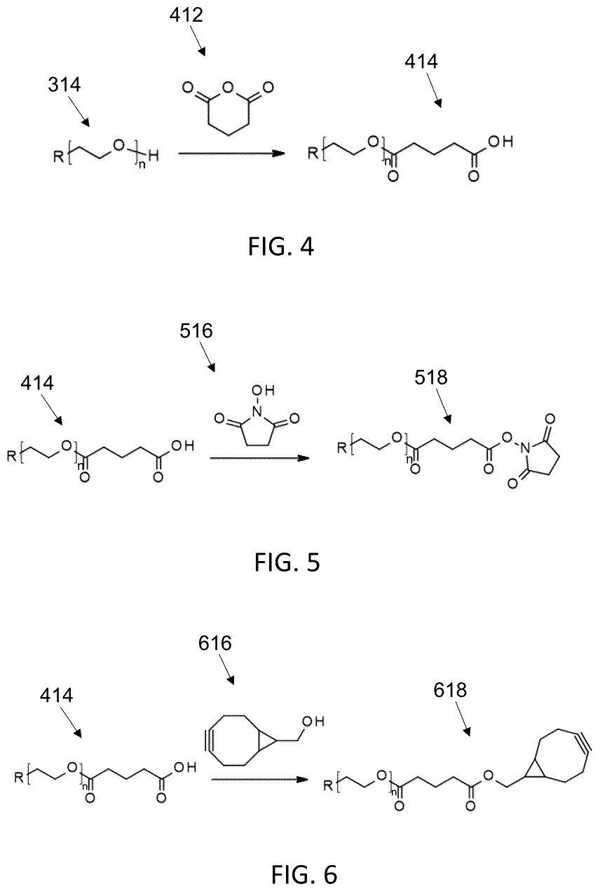

In a particular example shown in FIG. 4, a hydroxyl-terminated multi-arm polymer, specifically, the hydroxyl-terminated 8-arm PEG (314) of FIG. 3, where n is an integer representing the number of monomer units in the polymer segment shown and R represents a remainder of the molecule (including the trehalose residue core and the remaining 7 arms of the molecule) is reacted with glutaric anhydride (412) to yield a glutaric-acid-end-capped multi-arm polymer, specifically, a glutaric-acid-end-capped 8-arm PEG (414).

A reactive moiety may then be linked to the carboxylic-acid-terminated precursor polymer. For example, in particular embodiments where a cyclic imide ester group is employed as a reactive group, an N-hydroxy cyclic imide compound (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester group (e.g., a succinimide ester group, a maleimide ester group, a glutarimide ester group, a phthalimide ester group, a bicyclo[2.2.1]hept-5-ene-2,3-dicarboxylic acid imide ester group, etc.) that is linked to a polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, succinimidyl diglycolate groups, and succinimidyl 1,3-acetonedicarboxylate groups (1,3-acetonedicarboxylate groups may also be referred to herein as 3-oxopentanedioate groups), among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, bicyclo[2.2.1]hept-5-ene-2,3-dicarboxylic acid imidyl diglycolate groups, and bicyclo[2.2.1]hept-5-ene-2,3-dicarboxylic acid imidyl 1,3-acetonedicarboxylate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, maleimidyl diglycolate groups, and maleimidyl 1,3-acetonedicarboxylate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, and glutarimidyl 1,3-acetonedicarboxylate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, phthalimidyl diglycolate groups, and phthalimidyl 1,3-acetonedicarboxylate groups, among others.

In a particular example shown in FIG. 5, an acid end-capped multi-arm polymer, specifically, the glutaric-acid-end-capped 8-arm PEG (414) of FIG. 4, where n is an integer representing the number of monomer units in the polymer segment shown and R represents a remainder of the molecule (including the trehalose residue core and the remaining 7 arms of the molecule), is reacted with N-hydroxysuccinimide (516) to yield a multi-arm polymer having cyclic imide ester groups, specifically an 8-arm PEG having succinimidyl ester groups, more particularly, an 8-arm PEG (518) having succinimidyl glutarate groups.

In another particular example shown in FIG. 6, an acid end-capped multi-arm polymer, specifically, the glutaric-acid-end-capped 8-arm (PEG 414) of FIG. 4, where n is an integer representing the number of monomer units in the polymer segment shown and R represents a remainder of the molecule (including the trehalose residue core and the remaining 7 arms of the molecule), is reacted with (1R,8S,9s)-bicyclo[6.1.0]non-4-yn-9-ylmethanol (616) to yield a multi-arm polymer having the cyclic alkyne ester groups, particularly, an 8-arm PEG (618) having bicyclo[6.1.0]nonyne (BCN) glutarate groups.

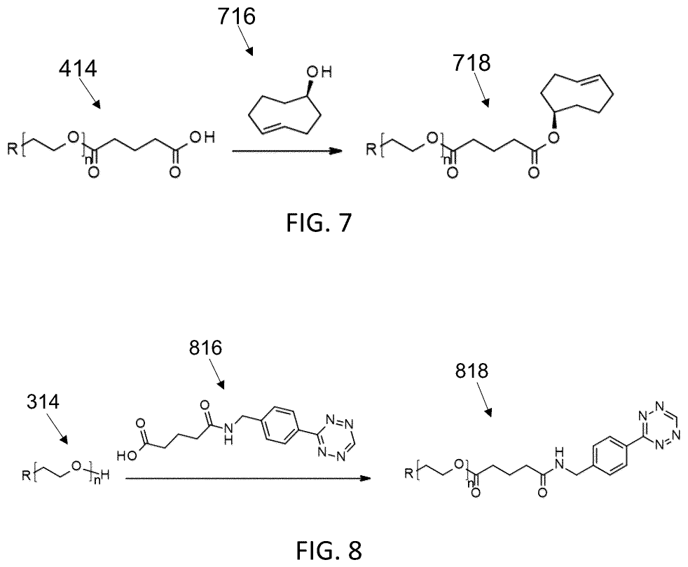

In another particular example shown in FIG. 7, an acid end-capped multi-arm polymer, specifically, the glutaric-acid-end-capped 8-arm PEG (414) of FIG. 4, where n is an integer representing the number of monomer units in the polymer segment shown and R represents a remainder of the molecule (including the trehalose residue core and the remaining 7 arms of the molecule), is reacted with a strained-alkene-containing alcohol, specifically, a cyclooctene-containing alcohol, more specifically, (E)-cyclooct-4-enol (716) to yield a multi-arm polymer having strained alkene ester groups, specifically, cyclooctene-containing ester groups, more specifically, an 8-arm PEG (718) having cyclooct-4-en-1-yl glutarate groups.

In a further particular example shown in FIG. 8, a hydroxyl-terminated multi-arm polymer, specifically, the hydroxyl-terminated 8-arm PEG (314) of FIG. 3, where n is an integer representing the number of monomer units in the polymer segment shown and R represents a remainder of the molecule (including the trehalose residue core and the remaining 7 arms of the molecule), is reacted with a tetrazine-containing carboxylic acid, specifically, 5-[4-(1,2,4,5-tetrazin-3-yl)benzylamino]-5-oxopentanoic acid (Bz-Tz-acid) (816) to yield a multi-arm polymer having tetrazine ester groups (818).

In some aspects, the present disclosure provides hydrogels that comprise a crosslinked reaction product of (a) a reactive multi-arm polymer comprising a plurality of first reactive moieties as described herein and (b) a multifunctional crosslinking compound having a plurality of second reactive moieties that are reactive with the first reactive moieties of the reactive multi-arm polymer.

In some embodiments, the hydrogels break down in-vivo over a period ranging from 2 days or less to 80 weeks or more, for example, ranging anywhere from 2 days to 4 days to 1 week to 2 weeks to 4 weeks to 10 weeks to 20 weeks to 40 weeks to 80 weeks (i.e., between any two or the preceding time periods).

In some aspects, the present disclosure provides systems that comprise (a) a reactive multi-arm polymer comprising a plurality of first reactive moieties as described herein and (b) a multifunctional crosslinking compound having a plurality of second reactive moieties that are reactive with the first reactive moieties of the reactive multi-arm polymer.

Multifunctional crosslinking compounds for use in the present disclosure may have three, four, five, six, seven, eight, nine, ten, eleven, twelve, thirteen, fourteen, fifteen, sixteen, seventeen, eighteen, nineteen, twenty, twenty-five, thirty or more second reactive moieties.

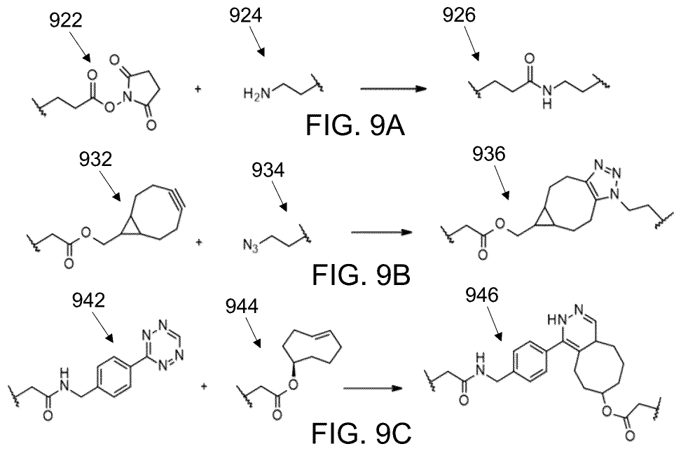

For example, with reference to FIG. 9A, a crosslinked reaction product can be formed from a reactive multi-arm polymer having electrophilic groups, for example, cyclic imide ester groups such as succinimide ester groups (922) and a multifunctional crosslinking compound having nucleophilic groups such as primary amine groups (924), which can be reacted with one another thereby forming amide-containing linkages (926). Conversely, a reactive multi-arm polymer having nucleophilic groups such as primary amine groups (924) and a multifunctional crosslinking compound having electrophilic groups, for example, cyclic imide ester groups such as succinimide ester groups (922) can be reacted with one another thereby forming amide-containing linkages (926).

As another example, with reference to FIG. 9B, a crosslinked reaction product can be formed from a reactive multi-arm polymer having strained alkyne groups, for example, cyclooctyne-containing groups such as bicyclo[6.1. 0]nonyne (BCN) groups (932), and a multifunctional crosslinking compound having azide groups (934), which can be reacted with one another thereby forming tricyclic linkages (936). Conversely, a reactive multi-arm polymer having azide groups (934) and a multifunctional crosslinking compound having strained alkyne groups (932) can be reacted with one another thereby forming tricyclic linkages (936).

As another example, with reference to FIG. 9C, a crosslinked reaction product can be formed from a reactive multi-arm polymer having tetrazine groups (942) and a multifunctional crosslinking compound having strained alkene groups, for example, cyclooct-4-en-1-yl groups (944), can be reacted with one another thereby forming cyclooctapyridazine linkages (946). Conversely, a reactive multi-arm polymer having strained alkene groups (944) and a multifunctional crosslinking compound having tetrazine groups (942) can be reacted with one another thereby forming cyclooctapyridazine linkages (946).

Additional crosslinked reaction products can be formed from the following: a reactive multi-arm polymer having diene groups and a multifunctional crosslinking compound having dienophilic groups; a reactive multi-arm polymer having dienophilic groups and a multifunctional crosslinking compound having diene groups; a reactive multi-arm polymer having alkene groups and a multifunctional crosslinking compound having thiol groups; a reactive multi-arm polymer having thiol groups and a multifunctional crosslinking compound having alkene groups; a reactive multi-arm polymer having oxyamine groups and a multifunctional crosslinking compound having aldehyde or ketone groups; and a reactive multi-arm polymer having aldehyde or ketone groups and a multifunctional crosslinking compound having oxyamine groups.

In some embodiments, hydrolysable moieties that comprises a hydrolysable linkage may be provided within the multifunctional crosslinking compound at positions internal to the second reactive moieties of the multifunctional crosslinking compound (e.g., wherein the second reactive moieties are each linked to a remainder of the multifunctional crosslinking compound through the hydrolysable linkage). Hydrolysable linkages may be selected, for example, from ester linkages, carbonate linkages, and acid anhydride linkages.

In some embodiments, iodinated multifunctional crosslinking compounds may be employed to provide radiopacity. In some of these embodiments, the iodinated multifunctional crosslinking compounds are compounds that comprise three or more second reactive moieties that are reactive with the reactive moieties of the reactive multi-arm polymer and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the three or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

In some aspects of the present disclosure, systems are provided that are configured to deliver (a) reactive multi-arm polymer as described herein and (b) a multifunctional crosslinking compound as described herein. The reactive multi-arm polymer and multifunctional crosslinking compound are comingled under conditions such that first reactive moieties of the reactive multi-arm polymer react and form covalent bonds with the second reactive moieties of the multifunctional crosslinking compound. Such systems can be used to form crosslinked hydrogels, either in vivo or ex vivo.

In some aspects of the present disclosure, systems are provided that comprise (a) a first composition that comprises a reactive multi-arm polymer as described herein and (b) a second composition that comprises a multifunctional crosslinking compound as described herein. For example, the first and second compositions can be first and second fluid compositions that, when the first and second fluid compositions are mixed, covalent bonds form between the reactive multi-arm polymer and the multifunctional crosslinking compound, resulting in a crosslinked reaction product of the reactive multi-arm polymer and the multifunctional crosslinking compound.

In some embodiments, systems are provided that comprise (a) a first composition containing the reactive multi-arm polymer and the multifunctional crosslinking compound and (b) a second composition comprising an accelerant that accelerates a crosslinking reaction between the reactive multi-arm polymer and the multifunctional crosslinking compound. For example, the first composition may be a fluid composition in which the reactive multi-arm polymer and the multifunctional crosslinking compound are intermixed under conditions where crosslinking is suppressed between the first reactive moieties of the reactive multi-arm polymer and the second reactive moieties of the multifunctional crosslinking compound, and the second composition may be a fluid composition that, when mixed with the first fluid composition, causes covalent bonds to form between the reactive multi-arm polymer and the multifunctional crosslinking compound, resulting in a crosslinked reaction product of the reactive multi-arm polymer and the multifunctional crosslinking compound. In certain embodiments, the accelerant in the second fluid composition changes the pH of the first fluid composition, resulting in crosslinking between the reactive multi-arm polymer and the multifunctional crosslinking compound.

The first composition may be a first fluid composition or may be first dry composition to which a suitable fluid such as water for injection, saline, etc. can be added to form a first fluid composition. The second composition may independently be a second fluid composition or may be second dry composition to which a suitable fluid such as water for injection, saline, etc. can be added to form a second fluid composition. The first and second compositions may independently be provided in vials, syringes, or other reservoirs.

The first and second compositions may further comprise additional agents, including therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second fluid compositions to a subject. Preferred subjects include mammalian subjects, particularly human subjects.

In some embodiments, the system may include a delivery device that comprises a first reservoir that contains a first fluid composition that comprises a reactive multi-arm polymer as described above and a second reservoir that contains a second fluid composition that comprises a multifunctional crosslinking compound as described above. When the first and second fluid compositions are mixed, crosslinking occurs between the reactive multi-arm polymer and the multifunctional crosslinking compound.

In some embodiments, the system may include a delivery device that comprises a first reservoir that contains a first fluid composition that comprises the reactive multi-arm polymer and the multifunctional crosslinking compound, and a second reservoir that contains a second fluid composition that is an accelerant composition. The second fluid composition, when mixed with the first fluid composition, results in crosslinking between the reactive multi-arm polymer and the multifunctional crosslinking compound.

In either case, during operation, the first fluid composition and second fluid composition are dispensed from the first and second reservoirs and combined, whereupon the multifunctional crosslinking compound and the reactive multi-arm polymer and crosslink with one another to form a crosslinked hydrogel.

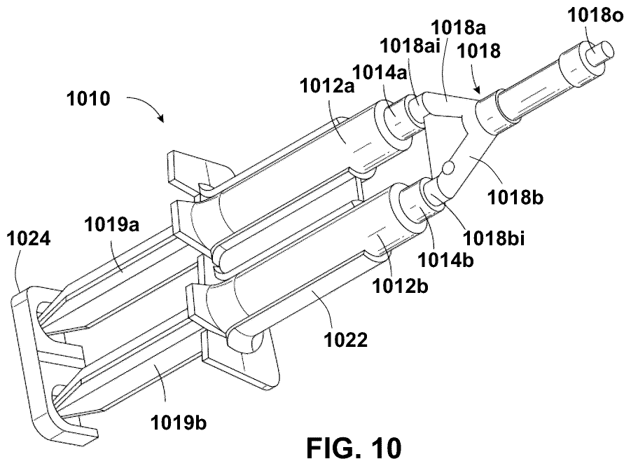

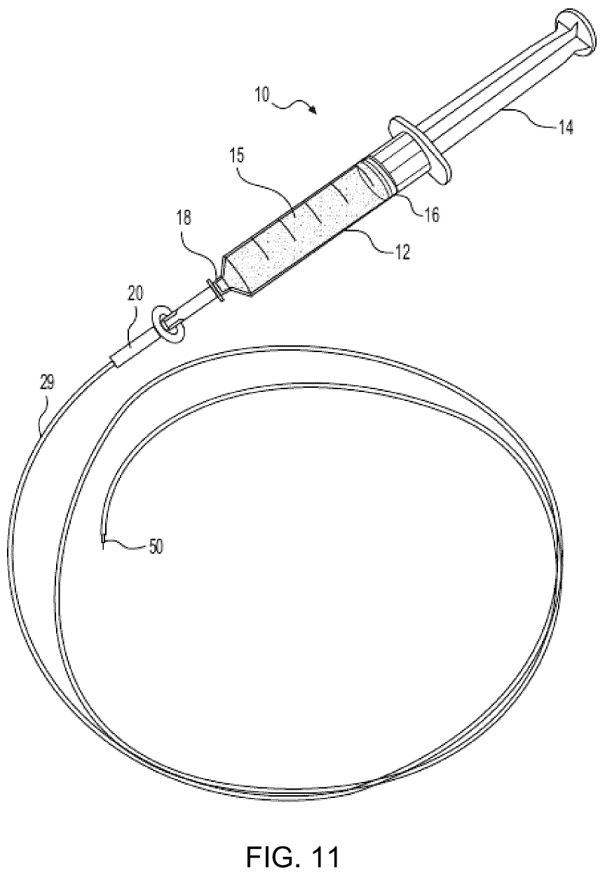

In particular embodiments, and with reference to FIG. 10, the system may include a delivery device 1010 that comprises a double-barrel syringe, which includes a first barrel 1012a having a first barrel outlet 1014a, which first barrel contains the first fluid composition, a first plunger 1016a that is movable in the first barrel 1012a, a second barrel 1012b having a second barrel outlet 1014b, which second barrel 1012b contains the second fluid composition, and a second plunger 1016b that is movable in the second barrel 1012b. In some embodiments, the device 1010 may further comprise a mixing section 1018 having a first mixing section inlet 1018ai in fluid communication with the first barrel outlet 1014a, a second mixing section inlet 1018bi in fluid communication with the second barrel outlet, and a mixing section outlet 1018o. Also shown are a syringe holder 1022 configured to hold the first and second syringe barrels 1012a, 1012b, in a fixed relationship and a plunger cap 1024 configured to hold the first and second plungers 1016a, 1016b in a fixed relationship.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture initially may be in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman@left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size.

Granulating may be by any suitable process, for instance by grinding (including cryogrinding), homogenization, crushing, milling, pounding, or the like. Sieving or other known techniques can be used to classify and fractionate the particles.

Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

Crosslinked hydrogel compositions in accordance with the present disclosure include injectable fluid suspensions of crosslinked hydrogel particles.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

FIG. 11 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

The crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

After administration, the crosslinked hydrogel compositions of the present disclosure can be imaged using a suitable imaging technique.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A reactive multi-arm polymer comprising (a) a core region, (b) multiple polymer arms, each polymer arm comprising at least one polymer segment and having a first end and a second end, the first end linked to the core region and (c) at least one first reactive moiety linked to the second end of each of the polymer arms, wherein the core region comprises a residue of a saccharide-based compound in which hydroxyl groups of a monosaccharide, disaccharide or oligosaccharide are replaced by C₃-C₁₀-hydroxyalkyloxy groups.

2. The reactive multi-arm polymer of claim 1, wherein the C₃-C₁₀-hydroxyalkyloxy groups are C₃-C₁₀-monohydroxyalkyloxy groups.

3. The reactive multi-arm polymer of claim 2, wherein the C₃-C₁₀-monohydroxyalkyloxy groups are linear C₃-C₁₀-monohydroxyalkyloxy groups and wherein a hydroxyl group is positioned at a terminal carbon atom of each of the linear C₃-C₁₀-monohydroxyalkyloxy groups.

4. The reactive multi-arm polymer of claim 1, wherein the C₃-C₁₀-hydroxyalkyloxy groups are C₃-C₁₀-dihydroxyalkyloxy groups.

5. The reactive multi-arm polymer of claim 4, wherein the C₃-C₁₀-dihydroxyalkyloxy groups are linear C₃-C₁₀-dihydroxyalkyloxy groups having two hydroxyl groups and wherein the two hydroxyl groups are vicinal diols with a first hydroxyl group positioned at a terminal carbon of each of the linear C₃-C₁₀-dihydroxyalkyloxy groups and a second hydroxyl group positioned at a carbon atom adjacent to the terminal carbon atom.

6. The reactive multi-arm polymer of claim 1, wherein the first reactive moieties are linked to the polymer segments through a hydrolysable linkage.

7. The reactive multi-arm polymer of claim 1, wherein the first reactive moiety is selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, reactive moieties that comprise oxyamine groups, and reactive moieties that comprise acrylate groups.

8. The reactive multi-arm polymer of claim 1, wherein the polymer segment is selected from polyalkylene oxide segments, polyester segments, polyoxazoline segments, polydioxanone segments, and polypeptide segments.

9. The reactive multi-arm polymer of claim 1, wherein the polymer segment contains between 10 and 1000 monomer residues.

10. A system for forming a hydrogel composition that comprises (a) a reactive multi-arm polymer comprising (i) a core region, (ii) multiple polymer arms, each polymer arm comprising at least one polymer segment and having a first end and a second end, the first end linked to the core region and (iii) at least one first reactive moiety linked to the second end of each of the polymer arms, wherein the core region comprises a residue of a saccharide-based compound in which hydroxyl groups of a monosaccharide, disaccharide or oligosaccharide are replaced by C₃-C₁₀-hydroxyalkyloxy groups and (b) a multifunctional crosslinking compound that comprises a plurality of second reactive moieties that are reactive with the first reactive moieties of the reactive multi-arm polymer.

11. The system of claim 10, wherein the plurality of second reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, reactive moieties that comprise oxyamine groups, and reactive moieties that comprise acrylate groups.

12. The system of claim 10, wherein the multifunctional crosslinking compound is an iodinated multifunctional crosslinking compound.

13. The system of claim 12, wherein the iodinated multifunctional crosslinking compound comprises an iodinated aromatic group.

14. The system of claim 10, further comprising a delivery device that is configured to simultaneously deliver the reactive multi-arm polymer and the multifunctional crosslinking compound to a patient under conditions where the reactive multi-arm polymer covalently crosslinks with the multifunctional crosslinking compound.

15. A crosslinked hydrogel formed by covalently crosslinking (a) a reactive multi-arm polymer comprising (i) a core region, (ii) multiple polymer arms, each polymer arm comprising at least one polymer segment and having a first end and a second end, the first end linked to the core region and (iii) at least one first reactive moiety linked to the second end of each of the polymer arms, wherein the core region comprises a residue of a saccharide-based compound in which hydroxyl groups of a monosaccharide, disaccharide or oligosaccharide are replaced by C₃-C₁₀-hydroxyalkyloxy groups and with (b) a multifunctional crosslinking compound that comprises a plurality of second reactive moieties that are reactive with the first reactive moieties of the reactive multi-arm polymer.

16. The crosslinked hydrogel of claim 15, wherein the plurality of second reactive moieties are selected from reactive moieties that comprise electrophilic groups, reactive moieties that comprise nucleophilic groups, reactive moieties that comprise diene groups, reactive moieties that comprise dienophile groups, reactive moieties that comprise alkenyl-containing groups, reactive moieties that comprise strained alkyne groups, reactive moieties that comprise azide groups, reactive moieties that comprise ketone groups, reactive moieties that comprise aldehyde groups, reactive moieties that comprise oxyamine groups, and reactive moieties that comprise acrylate groups.

17. The crosslinked hydrogel of claim 15, wherein the multifunctional crosslinking compound is an iodinated multifunctional crosslinking compound.

18. The crosslinked hydrogel of claim 17, wherein the iodinated multifunctional crosslinking compound comprises an iodinated aromatic group.

19. The crosslinked hydrogel of claim 15, wherein the crosslinked hydrogel is in the form of crosslinked hydrogel particles.

20. The crosslinked hydrogel of claim 19, wherein the crosslinked hydrogel particles are contained in a syringe barrel.
